(19) United States
(12) Patent Application Publication
(10) Pub. No.: US 2025/0257401 A1
(43) Pub. Date: Aug. 14, 2025

(54) BIOMARKER FOR TUBEROUS SCLEROSIS COMPLEX WITH NO MUTATION IDENTIFIED IN TSC1 OR TSC2 GENE, AND USES THEREOF

(71) Applicants: Guangzhou KingMed Transformative Medicine Institute Co., Ltd., Guangzhou City (CN); Guangzhou Kingmed Center for Clinical Laboratory, Guangzhou City (CN); Guangzhou Kingmed Diagnostics Group Co., Ltd., Guangzhou City (CN)

(72) Inventors: Yuhuan MENG, Guangzhou City (CN); Tao CHEN, Guangzhou City (CN); Xijie FAN, Guangzhou City (CN); Guibin LI, Guangzhou City (CN); Rumei SUN, Guangzhou City (CN); Xiaping MIAO, Guangzhou City (CN); Yating CHENG, Guangzhou City (CN); Shihui YU, Guangzhou City (CN); Yaoming LIANG, Guangzhou City (CN)

(21) Appl. No.: 18/555,745

(22) PCT Filed: Dec. 1, 2022
(86) PCT No.: PCT/CN2022/135789

§ 371 Date: Oct. 17, 2023

(30) Foreign Application Priority Data
Nov. 3, 2022 (CN) 202211368365.0

(51) Int. Cl. C12Q 1/6883

(52) U.S. Cl. CPC C12Q 1/6883; C12Q 2600/106; C12Q 2600/156

(57) ABSTRACT

A biomarker for Tuberous sclerosis complex (TSC) with no mutation identified (NMI) in the TSC1 or TSC2 gene and a use thereof is provided. The biomarker is the expression level of IQGAP2 gene. The present disclosure analyzed and compared the whole-exon sequencing/clinical whole-exon sequencing results of TSC-NMI (No Mutation Identified, i.e., tuberous sclerosis complex with no mutation identified (NMI) in the TSC1 or TSC2 gene) patients with those of TSC patients having pathogenic mutations in TSC1 and TSC2 gene, and found a number of potential pathogenic candidate genes for TSC-NMI. Based on the long-term accumulated experience and experimental validation in this field, it was finally found that the IQGAP2 gene is related to TSC-NMI. Thus, IQGAP2 gene can be detected, and used as a detection marker for TSC-NMI and has potential as a drug target for treating the TSC-NMI patients.

### CROSS-REFERENCE TO RELATED APPLICATIONS

This application claims priority to PCT Application No. PCT/CN2022/135789, having a filing date of Dec. 1, 2022, which is based on Chinese Application No. 202211368365.0, having a filing date of Nov. 3, 2022, the entire contents both of which are hereby incorporated by reference.

### SEQUENCE LISTING

This application includes a separate sequence listing in compliance with the requirement of WIPO Standard ST.26, submitted under the file name “Sequence Listing”, created on Oct. 16, 2023, having a file size of 4 KB, the contents of which are hereby incorporated by reference.

### FIELD OF TECHNOLOGY

The following relates to the technical field of biomedicine, and particularly, it relates to a biomarker for Tuberous sclerosis complex (TSC) with no mutation identified (NMI) in the TSC1 or TSC2 gene, and a use thereof.

### BACKGROUND

Tuberous sclerosis complex (TSC) is an uncommon genetic disorder that causes abnormal growth in nervus and skin, and it has been recorded in China's First National List of Rare Diseases. TSC is mainly characterized by multiple-system nodules or multiple organ hamartoma, which is usually found in organs, such as skin, brain and kidneys, with a prevalence of one in 10,000 to one in 6,000 individuals. TSC is an autosomal dominant genetic disorder, and is mainly caused by the mechanistic target of rapamycin complex 1 (mTORC1) overactivation due to aberrant TSC1 or TSC2 gene, and thus leads to excessive cell proliferation, thereby forming multiple-system nodules or multiple organ hamartomas. If pathogenic mutations are found in TSC1 or TSC2 genes through gene testing, the patients can be diagnosed with TSC.

However, 15% of TSC with no mutation identified (NMI) in the TSC1 or TSC2 gene, which is called TSC-NMI. TSC-NMI patients can be diagnosed by identifying phenotype characteristics based on the clinical diagnostic criteria only. According to the clinical diagnostic criteria of TSC, a patient having two major features or one major feature along with two minor features can be diagnosed with “definite TSC”, and a patient having one major feature or two minor features can be diagnosed with “possible TSC”. Most characteristics need to be detected by medical imaging, such as computerized tomography (CT), magnetic resonance imaging (MRI), and heart color ultrasound (UCG), etc., which increase the diagnosis cost and difficulty for TSC-NMI patients, thereby resulting in missed diagnosis and later diagnosis of TSC-NMI and missing out on early intervention and treatment.

### SUMMARY

An aspect relates to providing a biomarker for Tuberous sclerosis complex with no mutation identified) in the TSC1 or TSC2 gene, which can serve as a gene marker for detecting TSC, and particularly for detecting TSC-NMI.

The present disclosure discloses a biomarker for Tuberous sclerosis complex with no mutation identified in the TSC1 or TSC2 gene (TSC-NMI), wherein the biomarker is an IQ-motif containing GTPase activating protein 2 (IQGAP2) gene.

The whole-exon sequencing/clinical whole-exon sequencing results of TSC-NMI (No Mutation Identified, i.e., tuberous sclerosis complex (TSC) with no mutation identified in the TSC1 or TSC2 gene) patients with those of TSC patients having pathogenic mutations in TSC1 and TSC2 gene, has been analyzed and compared and a number of potential pathogenic candidate genes for TSC-NMI have been found. Based on the long-term accumulated experience and experimental validation in this field, it was finally found that the IQGAP2 gene was related to TSC-NMI. Thus, the IQGAP2 gene can be detected and used as a detection marker for TSC-NMI, and has a potential as a drug target for treating TSC-NMI patients.

In one example, when the IQGAP2 gene is mutated, silenced, or down-regulated, it is indicated that there is a risk for Tuberous sclerosis complex with no mutation identified (NMI) in the TSC1 or TSC2 gene.

It is another aspect to provide the use of the biomarker for diagnosing and/or treating Tuberous sclerosis complex with no mutation identified in the TSC1 or TSC2 gene.

It is another aspect to provide the use of the biomarker as a target in the preparation of a reagent for diagnosing Tuberous sclerosis complex with no mutation identified in the TSC1 or TSC2 gene or as a target in the preparation of a medicament for treating Tuberous sclerosis complex with no mutation identified in the TSC1 or TSC2 gene

It can be noted that, the term “target” refers to a direct or indirect detection subject, including the IQGAP2 gene having detrimental mutations and its corresponding mRNAs or proteins, for evaluating the risk of TSC-NMI, i.e., IQGAP2 gene having the detrimental mutations and its corresponding mRNAs or proteins act as a detection marker for TSC-NMI. The target can achieve the same effect as TSC1 and TSC2 genes in detection, greatly reducing the difficulty of diagnosing TSC-NMI. It is also indicated that the IQGAP2 gene having the mutations and its corresponding mRNAs or proteins can be used as a drug target to inhibit AKT activity and/or inhibit mTOR activity, so as to suppress cell proliferation through gene editing, mRNA drug, or direct administration of large-molecule protein drugs, thereby achieving the purpose of treating tuberous sclerosis complex with no mutation identified (NMI) in the TSC1 or TSC2 gene.

In one example, the use of a reagent for detecting the biomarker in preparation of a reagent for diagnosing Tuberous sclerosis with no mutation identified (NMI) in the TSC1 or TSC2 gene is provided.

In one example, use of an IQGAP2 gene activator in the preparation of a medicament for treating tuberous sclerosis complex with no mutation identified (NMI) in the TSC1 or TSC2 gene is provided.

In one example, the IQGAP2 gene activator suppresses cell proliferation by inhibiting AKT activity and/or inhibiting mTOR activity, thereby treating tuberous sclerosis complex with no mutation identified in the TSC1 or TSC2 gene.

It is another aspect to provide a kit for assisting in diagnosing tuberous sclerosis complex with no mutation identified in the TSC1 or TSC2 gene, comprising a reagent for detecting IQGAP2 gene.

It can be noted that, the above-mentioned reagent for detecting IQGAP2 gene comprises a reagent for detecting gene mutation or expression level of IQGAP2 gene.

It is another aspect to provide a non-diagnosis and non-treatment gene detection method for Tuberous sclerosis complex with no mutation identified in the TSC1 or TSC2 gene, comprising steps of detecting a gene mutation and/or an expression level of IQGAP2 gene in a biological sample and determining a risk of Tuberous sclerosis complex with no mutation identified in the TSC1 or TSC2 gene based on the detection results.

It is another aspect to provide a system of detecting Tuberous sclerosis complex with no mutation identified in the TSC1 or TSC2 gene, comprising following modules:
a detection module, configured for detecting a gene mutation or an expression level of IQGAP2 gene in a biological sample; and
an analysis module, configured for obtaining a detection result from the detection module, and comparing the detection result with a pre-determined value, wherein when a detrimental mutation occurs in IQGAP2 gene or the expression level of IQGAP2 gene is less than the pre-determined value, it is indicated that there is a high risk of Tuberous sclerosis complex with no mutation identified in the TSC1 or TSC2 gene.

It can be noted that, through conventional methods in this field, the above-mentioned pre-determined value can be obtained by collecting a plurality of samples, then comparing and analyzing TSC-NMI patients with TSC1/TSC2 mutations TSC patients (TSC patients), as well as conventional parameters including detection rate.

Compared to the conventional art, embodiments of the present disclosure have the advantages as follows:

A biomarker for Tuberous sclerosis complex with no mutation identified (NMI) in the TSC1 or TSC2 gene of the present disclosure, which indicates the expression level of IQGAP2 gene, is obtained, through analyzing and comparing the whole-exon sequencing/clinical whole-exon sequencing results of TSC-NMI patients with those of TSC patients having TSC1 and TSC2 pathogenic mutations, exploring a plurality of potential pathogenic candidate genes, and finally finding that the IQGAP2 gene is related to TSC-NMI, based upon the long-term accumulated experience and experimental validation in this field. Thus, the IQGAP2 gene can be detected and act as a detection marker for TSC-NMI and has potential as a drug target for treating TSC-NMI patients.

Using the biomarker of the present disclosure, TSC-NMI patients can be diagnosed by only detecting the abnormal IQGAP2 gene. This greatly reduces the difficulty and time of diagnosing TSC-NMI and is beneficial for early intervention and treatment to gain survival time for the patients.

### BRIEF DESCRIPTION

Some of examples will be described in detail, with reference to the following figures, wherein like designations denote like members, wherein:

FIG. 1 shows the potential pathogenic candidate gene for TSC-NMI obtained from the mutation load analysis of detrimental mutations in Example 1;

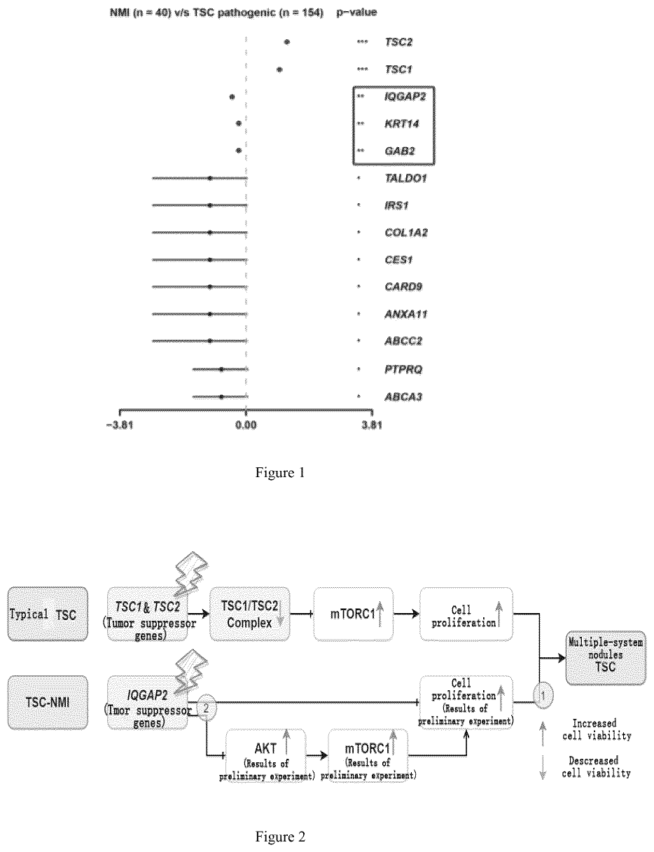

FIG. 2 shows a speculated mechanism of TSC-NMI caused by abnormal IQGAP2 in Example 1;

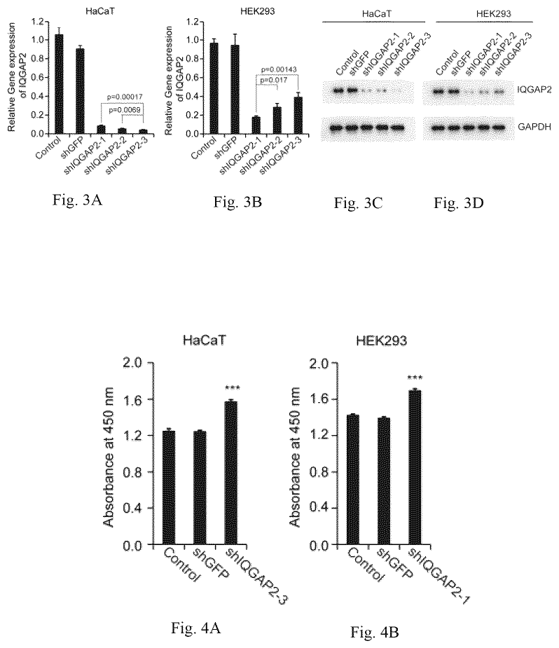

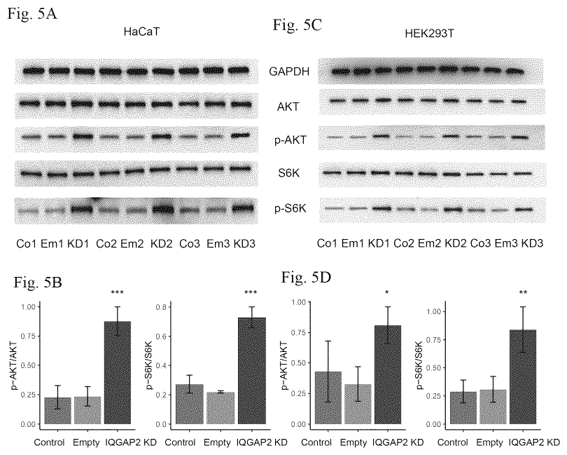

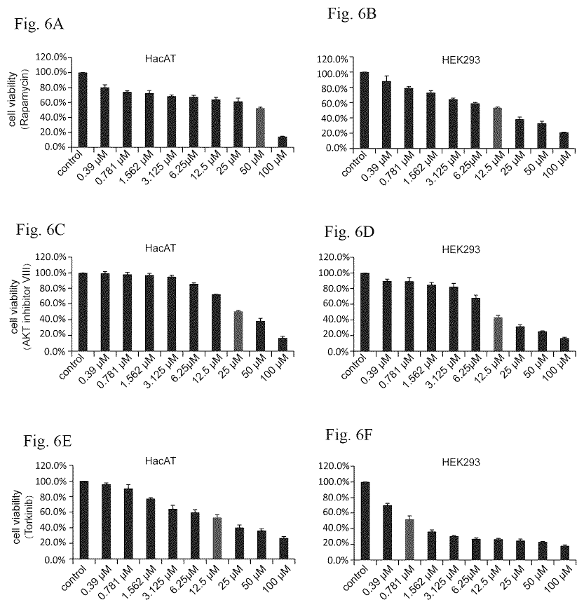

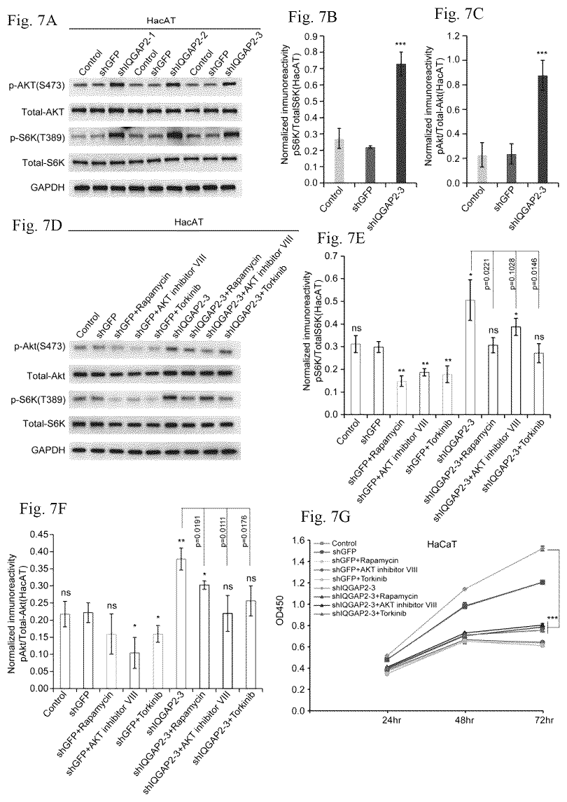

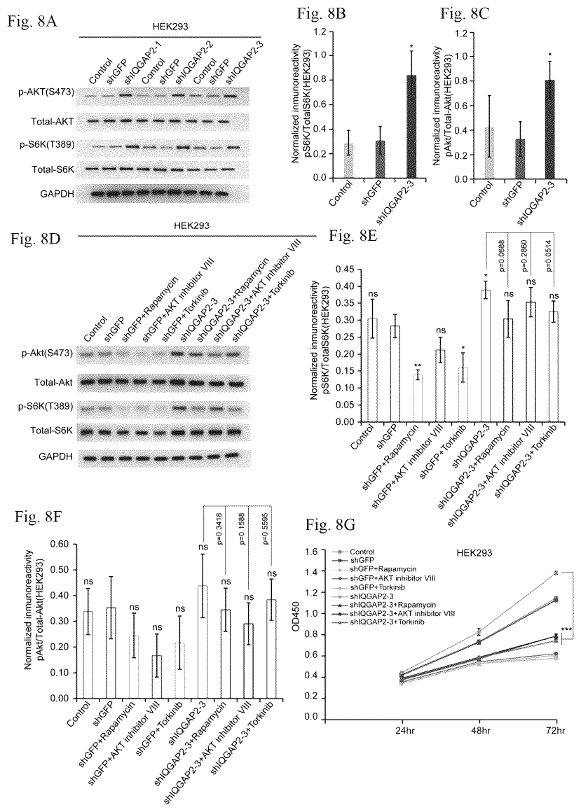

FIG. 3A shows the gene expression level of IGQGAP2-silenced human keratinocytes (HaCaT) cells in Example 2;

FIG. 3B shows shows the gene expression level of IGQGAP2-silenced human embryonic kidney 293 (HEK 293) cells in Example 2;

FIG. 3C shows shows the gene expression level of IGQGAP2-silenced human keratinocytes (HaCaT) cells in Example 2;

FIG. 3D shows shows the gene expression level IGQGAP2-silenced human embryonic kidney 293 (HEK 293) cells in Example 2;

FIG. 4A shows cell proliferation of IGQGAP2-silenced human keratinocytes cells in Example 2;

FIG. 4B shows cell proliferation of IGQGAP2-silenced human embryonic kidney 293 cells in Example 2;

FIG. 5A is a schematic diagram showing ATK activity and mTORC1 activity in IGQGAP2-silenced HaCaT cells in Example 2;

FIG. 5B is a schematic diagram showing ATK activity and mTORC1 activity in IGQGAP2-silenced HaCaT cells in Example 2;

FIG. 5C is a schematic diagram showing ATK activity and mTORC1 activity in IGQGAP2-silenced HEK 293 cells in Example 2;

FIG. 5D is a schematic diagram showing ATK activity and mTORC1 activity IGQGAP2-silenced HEK 293 cells in Example 2;

FIG. 6A shows the results of dose-dependent experiments on the cell viability of HaCaT cells cells after administration of an AKT inhibitor and mTOR inhibitors in Example 2;

FIG. 6B shows the results of dose-dependent experiments on the cell viability of HEK 293 cells after administration of an AKT inhibitor and mTOR inhibitors in Example 2;

FIG. 6C shows the results of dose-dependent experiments on the cell viability of HaCaT cells after administration of an AKT inhibitor and mTOR inhibitors in Example 2;

FIG. 6D shows the results of dose-dependent experiments on the cell viability of HEK 293 cells after administration of an AKT inhibitor and mTOR inhibitors in Example 2;

FIG. 6E shows the results of dose-dependent experiments on the cell viability of HaCaT cells after administration of an AKT inhibitor and mTOR inhibitors in Example 2;

FIG. 6F shows the results of dose-dependent experiments on the cell viability of HEK 293 cells after administration of an AKT inhibitor and mTOR inhibitors in Example 2;

FIG. 7A shows the cell proliferation of IGQGAP2-silenced HaCaT cells after administration of an AKT inhibitor and mTOR inhibitors in Example 2;

FIG. 7B shows the cell proliferation of IGQGAP2-silenced HaCaT cells after administration of an AKT inhibitor and mTOR inhibitors in Example 2;

FIG. 7C shows the cell proliferation of IGQGAP2-silenced HaCaT cells after administration of an AKT inhibitor and mTOR inhibitors in Example 2;

FIG. 7D shows the cell proliferation of IGQGAP2-silenced HaCaT cells after administration of an AKT inhibitor and mTOR inhibitors in Example 2;

FIG. 7E shows the cell proliferation of IGQGAP2-silenced HaCaT cells after administration of an AKT inhibitor and mTOR inhibitors in Example 2;

FIG. 7F shows the cell proliferation of IGQGAP2-silenced HaCaT cells after administration of an AKT inhibitor and mTOR inhibitors in Example 2;

FIG. 7G shows the cell proliferation of IGQGAP2-silenced HaCaT cells after administration of an AKT inhibitor and mTOR inhibitors in Example 2;

FIG. 8A shows cell proliferation of IGQGAP2-silenced HEK 293 cells after administration of an AKT inhibitor and mTOR inhibitors in Example 2;

FIG. 8B shows cell proliferation of IGQGAP2-silenced HEK 293 cells after administration of an AKT inhibitor and mTOR inhibitors in Example 2;

FIG. 8C shows cell proliferation of IGQGAP2-silenced HEK 293 cells after administration of an AKT inhibitor and mTOR inhibitors in Example 2;

FIG. 8D shows cell proliferation of IGQGAP2-silenced HEK 293 cells after administration of an AKT inhibitor and mTOR inhibitors in Example 2;

FIG. 8E shows cell proliferation of IGQGAP2-silenced HEK 293 cells after administration of an AKT inhibitor and mTOR inhibitors in Example 2;

FIG. 8F shows cell proliferation of IGQGAP2-silenced HEK 293 cells after administration of an AKT inhibitor and mTOR inhibitors in Example 2; and

FIG. 8G shows cell proliferation of IGQGAP2-silenced HEK 293 cells after administration of an AKT inhibitor and mTOR inhibitors in Example 2;

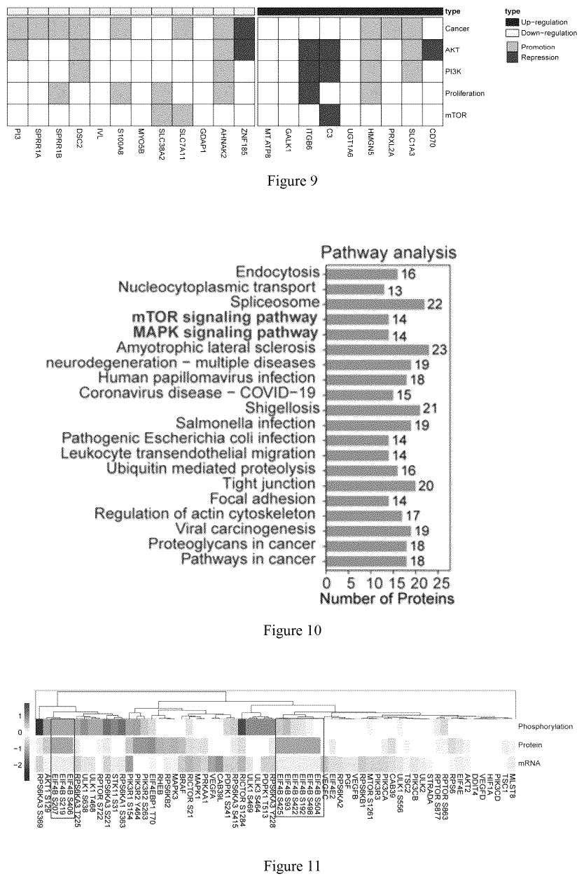

FIG. 9 shows both up-regulated and down-regulated genes in the transcriptome and proteome in Example 2;

FIG. 10 shows KEGG pathway enrichment analysis showing no changes in proteome but significant differences in phosphoproteome in Example 2; and

FIG. 11 shows the analysis results of mRNAs, proteins and phosphorylated sites in the mTOR pathway in Example 2.

### DETAILED DESCRIPTION

For a better understanding of the present disclosure, the present disclosure will be fully described below in reference to relevant drawings. Some preferred embodiments of the present invention are given in the drawings. However, the present disclosure can be implemented in many different forms and is not limited to the embodiments described herein. Rather, these embodiments are provided for the purpose of making the disclosed contents of the present disclosure more thorough and complete.

Unless otherwise defined, all technical and scientific terms used herein have the same meaning as those normally understood by one skilled in the conventional art in the technical field that the present disclosure is belonged to. The terms used in the description of the present disclosure herein are only for the purpose of describing specific embodiments, and are not intended to limit the present disclosure. The term “and/or” used herein refers to any one or more relevant items and their combinations.

The raw materials used in the following Examples are commercially available unless otherwise stated. In embodiments, the methods used in the following Examples are conventional methods, unless otherwise specified.

### EXAMPLE 1

### Screening Out TSC-NMI Pathogenic Genes

In the previous study, the results of whole-exon sequencing/clinical whole-exon sequencing were collected from 347 TSC-suspected patients and the pathogenicity of all mutations in the TSC1 and TSC2 genes was evaluated by ACMG guidelines. On this basis, TSC-suspected patients were diagnosed and classified according to guidelines on the TSC diagnostic combined with phenotype characteristics of TSC. And 169 patients with a definite diagnosis of TSC, 87 patients with a possible diagnosis of TSC, 25 patients with an uncertain diagnosis of TSC were obtained, and 66 patients with incomplete information were excluded. There were 40 TSC-NMI patients, accounting for 15.625% of patients with a definite diagnosis or possible diagnosis of TSC.

Using the detrimental mutation loads, all genes carrying detrimental mutations in TSC-NMI patients were then compared with those in TSC patients having pathogenic mutations in TSC1 and TSC2 genes, and the method comprised the following steps:
(1) screening for detrimental mutations, i.e., screening out the mutations that had a public database frequency of less than 0.01 and possibly altered amino acids, wherein the mutations included missense mutation, frameshift mutation caused by insertion and deletion and non-frameshift mutation, stopgain mutation, stoploss mutation, and splicing mutation, etc.
(2) classifying TSC-NMI patients as an experimental group, TSC patients with pathogenic mutations in TSC1 and TSC2 genes as a control group, and performing statistical test for detrimental mutations in each gene (including TSC1 and TSC2 genes);
(3) adopting Fisher's Exact test to determine statistical difference for detrimental mutations in each gene between the two groups, wherein when a p-value≤0.05, the difference was qualified as a significant difference.

The results were shown in FIG. 1, and several TSC-NMI potential pathogenic candidate genes, including IQGAP2, KRT14 and GAB2, were found. FIG. 1 was a forest graph of gene mutations, wherein the negative value below zero of the abscissa represented OR value (OR value referred to odds ratio) of the TSC-NMI group, the positive value above zero of the abscissa represented OR value of the TSC group, and OR value was odds ratio. The greater the absolute value for OR value was, the higher the incidence of TSC or TSC-NMI affected by the gene was. “*” of p-value represented p≤0.05 between the data of two groups, “**” represented p≤0.01 between the data of two groups, “***” represented p≤0.001 between the data of two groups.

It was found from previous experience and studies that IQGAP2 was related to the AKT activity. Overexpression of IQGAP2 could dephosphorylate AKT at the S473 site and significantly reduce the AKT activity. The IQGAP2 silencing could activate the AKT protein by inhibiting the activity of SH2 domain-containing inositol phosphatase 2 (SHIP2), i.e., a phosphatase, which increased epithelial-mesenchymal transition (EMT) and promoted cell migration and invasion. This showed that IQGAP2 and AKT activity had a negative correlation.

In view of the fact that ATK could inhibit the activity of the TSC1/TSC2 complex, it was hypothesized that IQGAP2 may be upstream of TSC1 and TSC2 genes and share the same function as that of TSC1 and TSC2 gene. Furthermore, in view of the fact that ATK could inhibit the activity of the TSC1/TSC2 complex, ATK and mTORC1 activity had a positive correlation. Thus, it was speculated that IQGAP2 may influence the ATK activity to further affect the mTORC1 activity.

Based on the above study, the IQGAP2 gene was identified as one of the potential pathogenic genes for TSC-NMI. Moreover, IQGAP2 shared the following three function characteristics with TSC1 and TSC2 genes: they were all tumor suppressor genes; their abnormal functions all could lead to excessive cell proliferation; and their abnormal functions all could lead to overactive mTORC1. Therefore, it was hypothesized that the abnormal IQGAP2 might cause excessive cell proliferation by mediating overactive mTORC1, and can be one of the important pathogenic genes leading to TSC-NMI, and the mechanism was shown in FIG. 2.

### EXAMPLE 2

### Validation on Function of IQGAP2 Gene

### 1. IQGAP2 Silencing can Lead to Excessive Cell Proliferation

To verify whether the abnormal IQGAP2 affected cell proliferation, IQGAP2-silenced cells were constructed using an RNAi approach. Since TSC could affect the skin and kidney of the patients, human keratinocytes (HaCaT) cells and human embryonic kidney 293 (HEK 293) cells were chosen as cell models. The shRNA sequence was designed and constructed for the IQGAP2 gene (as shown in Table 1), and IQGAP2-silenced HaCaT cells and IQGAP2-silenced HEK293 cells were obtained by lentiviral transduction. Subsequently, the cell proliferation of IQGAP2-silenced cells was detected at 24, 48, and 72 hours by using Cell Counting Kit 8 (CCK8).

TABLE 1
IQGAP2-silenced shRNA sequence

| | |
|---|---|
| IQGAP2-shRNA-1 | 5′-GCTCCTACCTACTGCGAATAT-3′ |
| | (SEQ ID NO. 1) |
| IQGAP2-shRNA-2 | 5′-GGGAAGAAGTAGTGACCAAGA-3′ |
| | (SEQ ID NO. 2) |
| IQGAP2-shRNA-3 | 5′-GCTCCAGATGGCTTTGATATC-3′ |
| | (SEQ ID NO. 3) |

The results were shown in FIGS. 3 to 4, and FIG. 3 depicted the gene expression level of IQGAP2-silenced human keratinocytes cells and IQGAP2-silenced human embryonic kidney 293 cells at 72 hours. In this figure, FIG. 3A showed the relative gene expression of IQGAP2 in HaCaT cells, FIG. 3B showed the relative gene expression of IQGAP2 in HEK 293 cells, FIG. 3C and FIG. 3D showed the electropherograms of IQGAP2 protein expression in HaCaT cells and in HEK 293 cells, respectively. And Control represented a control group, shGFP represented the empty vector control group, sh IQ IQGAP2-1, sh IQ IQGAP2-2, and sh IQ IQGAP2-3 represented the corresponding shRNA-1 to shRNA-3 primer silencing groups (repeated in triplicate for each group), respectively, and GAPDH represented the internal reference gene. The above results indicated that models of IQGAP2-silenced HaCaT and HEK293 cells were successfully constructed in this Example. Further, the above results also indicated that IQGAP2-shRNA-3 primer had optimum silencing in HaCaT cells, while IQGAP2-shRNA-1 primer had optimum silencing in HEK293 cells.

FIG. 4 showed the cell proliferation of IQGAP2-silenced human keratinocytes (HaCaT) cells and IQGAP2-silenced human embryonic kidney 293 (HEK 293) cells at 72 hours. In this figure, FIG. 4A showed the cell proliferation of HaCaT cells, FIG. 4B showed the cell proliferation of HEK 293 cells, Control represented a control group, shGFP represented the empty vector control group, sh IQ IQGAP2-1 and sh IQ IQGAP2-3 represented the corresponding shRNA primer silencing groups (repeated in triplicate for each group), respectively.

The above results showed that the IQGAP2-silenced HaCaT cells were more proliferative than the cells in the control group, with a cell proliferation rate of up to 26.04% at 72 hours; the above results also showed that IQGAP2-silenced HEK 293 cells were more proliferative than the cells in the control group, with a cell proliferation rate of up to 20.68% at 72 hours.

In conclusion, the test results showed that IQGAP2 silencing can greatly enhance cell proliferation.

2. IQGAP2 Silencing can Enhance the Activity of mTORC1

Based upon the existing test results, it was hypothesized that IQGAP2 possibly affected the mTORC1 activity by impacting on the ATK activity. In order to verify such hypothesis, the present Example further investigated the effect of IQGAP2 silencing on the ATK activity and mTORC1 activity. The ATK activity could be determined by the ratio of phosphorylated ATK (p-AKT) at S473 site to total AKT, and the mTORC1 activity could be determined by the ratio of phosphorylated S6K (p-S6K) at Thr389 site to total S6K.

Therefore, the amounts of p-AKT (phosphorylated ATK), total AKT, p-S6K (phosphorylated S6K), and total S6K of IQGAP2-silenced cells were detected respectively by using the immunoblotting (Western Blot), and then their changes in activities were calculated. An increase in the ratio of p-AKT to total AKT indicated an increase in ATC activity, and an increase in the ratio of p-S6K to total S6K indicated an increase in S6K activity.

The test results were shown in FIG. 5, which was a schematic graph showing ATK activity and mTORC1 activity in IQGAP2-silenced HaCaT cells and IQGAP2-silenced HEK283 cells. In the figure, FIG. 5A and FIG. 5C showed protein electropherograms of GAPDH (reference gene), total AKT, p-AKT, total S6K, and p-S6K of HaCaT cells and HEK283 cells, respectively, wherein in the abscissa, Co1, Em1, KD1, Co2, Em2, KD2, Co3, Em3, and KD3 respectively represented Control Group 1, Empty vector Control Group 1, IQGAP2-silenced Group 1 (IQGAP2-shRNA-1), Control Group 2, Empty vector Control Group 2, IQGAP2-silenced Group 2 (IQGAP2-shRNA-2), Control Group 3, Empty vector Control Group 3, and IQGAP2-silenced Group 3 (IQGAP2-shRNA-3); FIG. 5B and FIG. 5D respectively showed the ratio of p-AKT to total AKT in HaCaT cells and the ratio of p-S6K to total S6K in HEK293 cells, respectively; Control represented the control group, Empty represented the empty vector control group, IQGAP2 KD represented the IQGAP2-silenced group (wherein HaCaT cells were silenced with the IQGAP2-shRNA-3 primer, and HEK293 cells were silenced with IQGAP2-shRNA-1, the tests were repeated in triplicate for each group).

The test results showed that IQGAP2 silencing significantly enhanced AKT activity and mTORC1 activity.

3. The Cell Proliferation of IQGAP2-Silenced Cells was Reduced by Using a AKT Inhibitor (AKT Inhibitor VIII) and mTOR Inhibitors (Torkinib and Rapamycin)

Based upon the above test results, it was hypothesized that IQGAP2 silencing could lead to increased cell proliferation through activating AKT activity and mTORC1 activity. To further verify the pathway, a AKT inhibitor (AKT inhibitor VIII) and mTOR inhibitors (Torkinib and Rapamycin) were used to treat IQGAP2-silenced human keratinocytes (HaCaT cells) and IQGAP2-silenced HEK293 cells, respectively. That is, the pathway was further determined by verifying whether the cell proliferation caused by IQGAP2 silencing can be alleviated by inhibiting the AKT activity and mTORC1 activity (key factors of the mTOR pathway) in IQGAP2-silenced cells with the inhibitors.

Firstly, for determining the dose of each inhibitor, IC50 of each inhibitor was tested in a preliminary experiment prior to the experiment, and the results of the preliminary experiment were shown in FIG. 6. In this figure, FIG. 6A and FIG. 6B showed the effects of Rapamycin on the cell viability of HaCaT cells and HEK293 cells at different concentrations, respectively, as well as its IC₅₀ represented in grey. IC50 of Rapamycin was 50 μM for HaCaT cells, and IC₅₀ of Rapamycin was 12.5 μM for HEK293 cells. FIG. 6C and FIG. 6D showed the effects of AKT inhibitor VIII on the cell viability of HaCaT cells and HEK293 cells at different concentrations, respectively, as well as its IC₅₀ represented in grey. IC₅₀ of AKT inhibitor VIII was 25 μM for HaCaT cells, and IC₅₀ of AKT inhibitor VIII was 12.5 μM for HEK293 cells. FIG. 6E and FIG. 6F showed the effects of Torkinib on the cell viability of HaCaT cells and HEK293 cells at different concentrations, respectively, as well as its IC₅₀ represented in grey. IC₅₀ of Torkinib was 12.5 μM for HaCaT cells, and IC₅₀ of Torkinib was 0.781 μM for HEK293 cells.

Then, HaCaT cells and HEK293 cells were administrated with each inhibitor in a dose of IC₅₀. The results were shown in FIG. 7 (HaCaT cells) and FIG. 8 (HEK293 cells). FIG. 7 showed the cell proliferation of HaCaT cells after they were administrated with the AKT inhibitor and mTOR inhibitors. In the figure, FIG. 7A showed a pattern of western blotting assay of ATK activity and mTORC1 activity under the given detection conditions in the figure, wherein Control represented the control groups, shGFP represented the empty vector control groups, sh IQGAP2-1 to sh IQGAP2-3 represented the corresponding shRNA-1 to shRNA-3 primer silencing groups, respectively (repeated in triplicate for each group). FIG. 7A indicated that IQGAP2 silencing could enhance AKT activity and S6K activity. FIG. 7B was a bar chart showing western blotting assay of S6K activity normalized from FIG. 7A, and it indicated that IQGAP2 silencing can enhance S6K activity. FIG. 7C was a bar chart showing western blotting assay of AKT activity normalized from FIG. 7A, and it indicated that IQGAP2 silencing can enhance AKT activity. FIG. 7D showed western blotting assay of IQGAP2-silenced HaCaT cells after they were administrated with AKT inhibitor VIII, Torkinib and Rapamycin, wherein Control represented the control groups, shGFP represented the empty vector control groups, shGFP+Rapamycin represented the group in which the empty vector control group was administrated with Rapamycin, shGFP+Torkinib represented the group in which the empty vector control group was administrated with Torkinib, shGFP+AKT inhibitor VIII represented the group in which the empty vector control group was administrated with AKT inhibitor VIII, IQGAP2-3 represented IQGAP2 silencing group, IQGPA2-3+Rapamycin represented the group in which the IQGAP2 silencing group was administrated with Rapamycin, IQGPA2-3 +AKT inhibitor VIII represented the group in which the IQGAP2 silencing group was administrated with AKT inhibitor VIII, IQGPA2-3+Torkinib represented the group in which the IQGAP2 silencing group was administrated with Torkinib, and FIG. 7D indicated that AKT activity and mTORC1 activity were reduced after administration of the AKT inhibitor and mTORC1 inhibitors. FIG. 7E was a bar chart showing western blotting assay of S6K activity normalized from FIG. 7D, and it indicated that mTORC1 activity was reduced after administration of the AKT inhibitor and mTORC1 inhibitors. FIG. 7F was a bar chart showing western blotting assay of AKT activity normalized from FIG. 7D, and it indicated that AKT activity was reduced after administration of the AKT inhibitor and mTORC1 inhibitors. FIG. 7G showed the cell proliferation pattern at 24, 48, and 72 hours, and it showed that the cell proliferation of IQGAP2-silenced cells was alleviated after administration of ATK and mTORC1 inhibitors.

FIG. 8 showed the cell proliferation of HEK293 cells after administration of the AKT inhibitor and mTOR inhibitors, and the denotations and groups could be referred to FIG. 7.

The above results showed that the administration of AKT inhibitor (AKT inhibitor VIII) and mTOR inhibitors (Torkinib and Rapamycin) can reduce the cell proliferation of IQGAP2-silenced cells.

4. Multi-Omics, such as Transcriptome and Proteome, and Phosphorylated Proteome, Demonstrated that IQGAP2 Affected Cell Proliferation Through mTOR Pathway

A total of 12,829 genes expressed in transcriptome of IQGAP2-silenced human skin cells (i.e., IQGAP2-silenced HacaT cells) were detected by using RNA-seq technology. 362 up-regulated genes and 384 down-regulated genes were found by Bayes-regularized t-test, some of which were shown as follows.

TABLE 2
Some of up-regulated genes and down-regulated genes

| Up-regulated genes | Down-regulated genes |
|---|---|
| TNFRSF12A | CFH |
| KDM7A | WNT16 |
| MATK | TFPI |
| CEACAM7 | SLC7A2 |
| SYN1 | PDK4 |
| CELSR3 | FMO3 |
| MASP2 | AASS |
| CLCA4 | DCN |
| SNAI2 | ALOX5 |
| MYO16 | CYP24A1 |
| ADAMTS6 | CD74 |
| PER3 | PLEKHO1 |
| NNAT | HSD17B6 |
| HHAT | ANK1 |
| GALC | ADAM28 |

5,939 proteins and 18,000 phosphorylated sites within 4,163 proteins were detected, using LC-MS/MS mass spectrometry. There were 69 up-regulated differential proteins, 135 down-regulated differential proteins, 103 up-regulated differential phosphorylation sites and 187 down-regulated differential phosphorylation sites, some of which were shown as follows.

TABLE 3
Some differential proteins and differential phosphorylation sites

| | | Up-regulated | Down-regulated |
|---|---|---|---|
| Up-regulated | Down-regulated | differential | differential |
| differential | differential | phosphorylation | phosphorylation |
| proteins | proteins | sites | sites |
| HSPA6 | RRAS2 | SRRM2:2044:S | PDE8A:457:S |
| PI3 | NAA16 | PRPF40A:885:S | SRRM2:838:S |
| SPRR1A | INPP1 | ARHGAP23:677:S | PLAA:50:S |
| GPR107 | UCK2 | SRRM2:950:S | AFF4:814:S |
| KCTD9 | CENPV | H1-0:2:T | ZBTB41:57:S |
| SAMD4B | GPT2 | H1-1:2:S | AMER1:246:S |
| B3GALNT2 | CLPP | SEC16A:1371:S | TPD52:171:S |
| DEF6 | SPINT1 | METTL1:27:S | FNDC3B:208:S |
| GPATCH3 | PPIL2 | COBLL1:256:S | IK:417:S |
| SPRR1B | PCBD2 | IRS2:365:S | ZYX:344:S |
| S100A9 | HOOK3 | SPRR1B:11:T | NUDT5:3:S |
| DPYD | TCF25 | CEP170:466:S | ATN1:81:S |
| MED22 | IQGAP3 | THRAP3:535:S | EPB41L1:475:T |
| FAT1 | SLC38A5 | PLEKHG5:249:S | PKN1:562:S |
| RP9 | ITGAV | PKP1:155:S | VCL:434:S |

By comparing the differentially expressed genes and proteins in the transcriptome and proteome (see FIG. 9), it was found that 12 genes (PI3, SPRR1A, SPRR1B, DSC2, IVL, S100A8, MYO5B, SLC38A2, SLC7A11, GDAP1, AHNAK2 and ZNF185) were up-regulated in both transcriptome and proteome, and 9 genes (MT-ATP8, GALK1, ITGB6, C3, UGT1A6, HMGN5, PRXL2A, SLC1A3 and CD70) were down-regulated in both transcriptome and proteome. Most of the genes were related to tumor, cell proliferation, AKT, PI3K (AKT and PI3K are upstream of mTOR pathway) and mTOR pathway.

The results of integrative proteome and phosphoproteome were shown in FIG. 10. The KEGG enrichment analysis showed no change in proteome but significant difference in phosphoproteome, indicating that both mTOR pathway and MAPK signaling pathway affected the cell proliferation. Moreover, in the analysis results of mTOR pathway (see FIG. 11), eIF4B protein, the downstream protein of mTORC1, and most phosphorylated sites were up-regulated, indicating that the mTOR pathway was activated.

The above results showed that IQGAP2 impacted on cell proliferation through mTORC1 pathway.

In conclusion, it is verified from the above tests by the Examples that IQGAP2 can impact on the cell proliferation through AKT and mTORC1, and thereby affecting TSC-NMI.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A biomarker for Tuberous sclerosis complex with no mutation identified in the TSC1 or TSC2 gene, wherein the biomarker is IQGAP2 gene.

2. The biomarker of claim 1, wherein when IQGAP2 gene is mutated, silenced, or down-regulated, it is indicated that there is a risk for Tuberous sclerosis complex with no mutation identified in the TSC1 or TSC2 gene.

3. A method of diagnosing and/or treating Tuberous sclerosis complex with no mutation identified in the TSC1 or TSC2 gene, comprising applying the biomarker of claim 1.

4. A method of preparing a reagent for diagnosing Tuberous sclerosis complex with not mutation identified in the TSC1 or TSC2 gene, or a medicament for treating Tuberous sclerosis complex with no mutation identified in the TSC1 or TSC2 gene, comprising applying the biomarker of claim 1 as a target.

5. The method of claim 4, wherein the method comprises applying a reagent for detecting the biomarker in the preparation of the reagent for diagnosing Tuberous sclerosis complex with no mutation identified in the TSC1 or TSC2 gene.

6. The method of claim 4, wherein the method comprises applying an IQGAP2 gene activator in the preparation of the medicament for treating tuberous sclerosis complex with no mutation identified in the TSC1 or TSC2 gene.

7. The method of claim 6, wherein the IQGAP2 gene activator suppresses the cell proliferation by inhibiting AKT activity and/or inhibiting mTOR activity, thereby treating tuberous sclerosis complex with no mutation identified in the TSC1 or TSC2 gene.

8. A kit for assisting to diagnose tuberous sclerosis complex with no mutation identified in the TSC1 or TSC2 gene, comprising a reagent for detecting IQGAP2 gene.

9. A non-diagnosis and non-treatment gene detection method for tuberous sclerosis complex with no mutation identified in the TSC1 or TSC2 gene, comprising:
detecting gene mutation and/or expression level of IQGAP2 gene in a biological sample and determining a risk of Tuberous sclerosis complex with no mutation identified in the TSC1 or TSC2 gene based on the detection results.

10. A system of detecting Tuberous sclerosis complex with no mutation identified in the TSC1 or TSC2 gene, comprising following modules:
a detection module, configured for detecting a gene mutation or an expression level of IQGAP2 gene in a biological sample; and
an analysis module, configured for obtaining a detection result from the detection module, and comparing the detection result with a pre-determined value, wherein when a detrimental mutation occurs in IQGAP2 gene or the expression level of IQGAP2 gene is less than the pre-determined value, it is indicated that there is a high risk of Tuberous sclerosis complex with no mutation identified in the TSC1 or TSC2 gene.


SEQUENCE LISTING (3 sequences)

SEQ ID NO: 1 - RNA, 21 nt, synthetic construct
  source 1..21: mol_type = other RNA
gctcctacctactgcgaatat

SEQ ID NO: 2 - RNA, 21 nt, synthetic construct
  source 1..21: mol_type = other RNA
gggaagaagtagtgaccaaga

SEQ ID NO: 3 - RNA, 21 nt, synthetic construct
  source 1..21: mol_type = other RNA
gctccagatggctttgatatc